(19) United States
(12) Patent Application Publication
(10) Pub. No.: US 2026/0232378 A1
(43) Pub. Date: Aug. 13, 2026

(54) METHOD FOR MODELING A JOINT OF A PATIENT

(71) Applicant: TWINSIGHT, Grenoble (FR)

(72) Inventors: Marek Janusz BUCKI, Theys (FR); Mathieu Pierre Yves RIMAUD, Grenoble (FR); Antoine Pierre-André PERRIER, Yvry-sur-Seine (FR); Mathieu RaymondAlbert BALET, Grenoble (FR)

(73) Assignee: TWINSIGHT, Grenoble (FR)

(21) Appl. No.: 19/147,958

(22) PCT Filed: Jan. 23, 2024
(86) PCT No.: PCT/FR2024/050087

§ 371 Date: Jul. 14, 2025

(30) Foreign Application Priority Data
Jan. 23, 2023 (FR) 23/00597

(51) Int. Cl. A61B 34/10

(52) U.S. Cl. CPC A61B 34/10; A61B 2034/101; A61B 2034/105; A61B 2034/108

(57) ABSTRACT

A method for modeling a joint of a patient, the method including carrying out the steps of: (a) obtaining a candidate biomechanical model of the joint and a first set of medical images of the joint, representing the joint in a posture; (b) for at least one medical image of the first set, implementing at least one simulation of the candidate biomechanical model in which the joint has the same posture as in the medical image; (c) constructing an error model of the candidate biomechanical model by comparing, for at least one biomechanical metric of the joint and for at least one medical image of the first set for which at least one simulation has been implemented, the values of the biomechanical metric in the medical image and in the simulation(s); (d) validation or non-validation of the candidate biomechanical model on the basis of the error model.

### GENERAL TECHNICAL FIELD

The present invention relates to the field of biomechanics. More specifically, it concerns a method for modeling a joint of a patient for orthopedic applications.

### STATE OF THE ART

Before an intervention (particularly surgical) on a joint such as the knee, it is known to generate a biomechanical model of the joint, known as a «digital twin», to help the practitioner plan the intervention and optimize the therapeutic strategy. More specifically, the digital twin not only allows for the functional assessment of the joint, but also the customization of prostheses/orthoses, or the guidance of the surgical procedure.

The digital twin is reconstructed from 2D or 3D medical images of the joint, adapted to the problem to be treated.

The difficulty is that known techniques do not guarantee the accuracy of the digital twin (i.e., that it faithfully represents reality), and manual validation by an expert is required, by comparing metrics of interest measured on the subject with the corresponding metrics predicted by the model, see, for example, the document Validation of computational models in biomechanics; H B Henninger ↑, S P Reese, A E Anderson, and J A Weiss.

These manual validation procedures are complex, time-consuming, and error-prone, even when performed by an experienced practitioner. It is also known that medical data processing exhibits significant inter-operator variability, which undermines the objectivity sought by a validation procedure.

There is therefore a need for an automatic and reliable procedure for validating a digital twin of a joint that is efficient, objective, and versatile.

This invention improves the situation.

### PRESENTATION OF THE INVENTION

The present invention therefore relates, according to a first aspect, to a method for modeling a joint of a patient, the method being characterized in that it comprises the implementation, by data processing means of a server, of the steps of:
(a) Obtaining a candidate biomechanical model of said joint and a first set of medical images of said joint, representing said joint in a posture from a first set of postures, called validation images;
(b) For at least one medical image of said first set of medical images of said joint, implementing at least one simulation of the candidate biomechanical model in which said joint exhibits the same posture as in said medical image of said first set of images;
(c) Constructing an error model of the candidate biomechanical model by comparing, for at least one biomechanical metric of said joint and for at least one medical image of said first set of medical images of said joint for which at least one simulation has been performed, the values of said biomechanical metric in said medical image and in the simulation(s) performed for said medical image;
(d) Validating or not the candidate biomechanical model based on the error model.

According to advantageous and non-limiting features:

Wherein, the step (a) comprises sub-steps (a1) of obtaining said first set of medical images of said joint, as well as a second set of medical images of said joint, representing said joint in a posture from a second set of postures; and (a2) generating said candidate biomechanical model from the second set of medical images.

The first set of postures and the second set of postures are distinct.

The step (a1) also comprises obtaining a third set of medical images of said joint, representing said joint in a posture from a third set of postures; the step (a) comprising a sub-step (a3) of calibrating the candidate biomechanical model based on said third set of medical images.

The first set of postures and the third set of postures are distinct; and the second set of postures and the third set of postures are different but not necessarily distinct.

The step (a1) comprises acquiring the second set of medical images, and where appropriate the third set of medical images, by a medical imaging device.

The step (d) comprises calculating, for the biomechanical metric(s) and the medical image(s) of the first set, a confidence interval on the value of said biomechanical metric from the error model, and the candidate biomechanical model is not validated if a width of at least a given number of the calculated confidence intervals exceeds an uncertainty threshold of the metric.

The values of said biomechanical metric in said medical image and in the simulation(s) implemented for said medical image are calculated in step (c) based on the positions of characteristic anatomical points of said joint in said medical image and in the simulation(s) implemented for said medical image.

There is a plurality of characteristic anatomical points of said joint, and said biomechanical metric is a distance between two of said characteristic anatomical points of said joint or an angle defined by three of said characteristic anatomical points of said joint.

The method comprises a step (e) of revising the candidate biomechanical model if it is not validated, then repeating at least steps (b) to (d) on the basis of the revised biomechanical model as a new candidate biomechanical model.

The method comprises a step (f) of using the validated biomechanical model, comprising implementing at least one simulation of the validated biomechanical model in which said joint exhibits a target posture.

The step (f) comprises using said error model to evaluate a level of uncertainty regarding the result of said simulation of the validated biomechanical model.

Either said candidate biomechanical model is non-deterministic, and in step (b) for each medical image of said first set, a plurality of simulations is implemented; or said candidate biomechanical model is deterministic, and in step (b) for each medical image of said first set, a single simulation is implemented.

According to a second aspect, the invention concerns a server for modeling a joint of a patient, characterized in that it comprises data processing means configured to:
Obtain a candidate biomechanical model of said joint as well as a first set of medical images of said joint, representing said joint in a posture from a first set of postures, called validation images;
For at least one medical image of said first set of medical images of said joint, implement at least one simulation of the candidate biomechanical model in which said joint exhibits the same posture as in said medical image of said first set of images;
Construct an error model of the candidate biomechanical model by comparing, for at least one biomechanical metric of said joint and for at least one medical image of said first set of medical images of said joint for which at least one simulation has been performed, the values of said biomechanical metric in said medical image and in the simulation(s) performed for said medical image;
Validate or reject the candidate biomechanical model based on the error model.

According to a third aspect, the invention concerns a system comprising a server, according to the second aspect, and a medical imaging device, the data processing means being further configured to:
Obtain from said medical imaging device said first set of medical images of said joint, as well as a second set of medical images of said joint, representing said joint in a posture of a second set of postures;
generate said candidate biomechanical model from the second set of medical images.

According to a fourth and a fifth aspect, the invention concerns a computer program product comprising code instructions for executing a method according to the first aspect of modeling a joint of a patient; and a storage means readable by computer equipment on which a computer program product comprising code instructions for executing a method according to the first aspect of modeling a joint of a patient is recorded.

### PRESENTATION OF THE FIGURES

Other features and advantages of the present invention will become apparent upon reading the following description of a preferred embodiment. This description will be given with reference to the accompanying drawings, in which:

FIG. 1 is a diagram of a system for implementing the method according to the invention;

FIG. 2 is a flowchart illustrating the steps of an embodiment of the method according to the invention;

FIG. 3 illustrates the sets of postures used in an embodiment of the method according to the invention;

FIG. 4a schematically represents a first geometric model for calculating uncertainty intervals on a first example of a biomechanical metric;

FIG. 4b schematically represents a second geometric model for calculating uncertainty intervals on a second example of biomechanical metrics.

### DETAILED DESCRIPTION

### Architecture

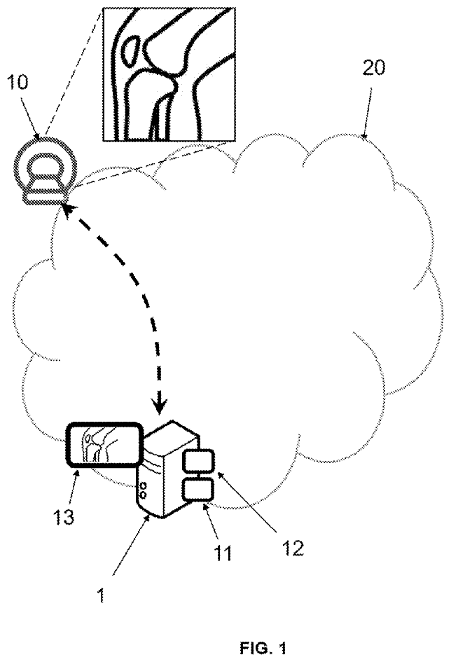

The present invention concerns a method for modeling a joint of a patient in a system represented in FIG. 1.

The joint, or jointure, is an anatomical element of the patient's body forming a junction zone between bone ends and forming at least one degree of freedom. Said joint 1 is typically the knee (which involves the femur, tibia, and patella), but it can also be the hip, ankle, elbow, wrist, shoulder, etc.). In particular, the joint can adopt a posture (or pose) among a plurality of possible postures (mathematically, Pₐₗₗ denotes the set of possible postures, see below), typically defined by one or more parameters such as angles, each corresponding to a possible movement of the joint. For example, the knee can thus produce the following movements:
Extension/flexion of the leg on the thigh (approximately 160° amplitude);
Internal/external rotation of the leg on the thigh (approximately 20° amplitude with the flexed knee);
Antero-posterior translation of the tibial plateau (approximately 10 mm).

The modeling of a joint requires, in particular, at least the validation of a biomechanical model of said joint (i.e., the evaluation of the model's ability to represent all or part of a real system within a predefined accuracy corridor—in this case, the joint of a patient). The method requires the prior generation of said model (or a model to be validated is provided). It is understood that the biomechanical model is in fact personalized, that is to say it is specifically adapted to this joint of a patient, and that the validation is for this joint and not another. A «global» model, i.e., that could be transposed to all patients, cannot be sufficiently accurate (and would therefore not be validated by this method).

For convenience, we refer to the «candidate» biomechanical model before validation, and then to the «validated» biomechanical model for the joint of a patient if the validation is successful, i.e., the candidate biomechanical model is not rejected. In a preferred embodiment, the rejected model can be «revised», and a new candidate model is obtained, which will itself be subjected to a validation, etc.

Thus, the output of the method is generally a validated biomechanical model, but in some cases, it may be an information indicating that a satisfactory biomechanical model was not obtained (if the method does not provide for revision or if it proves impossible, see below).

In all cases, by «biomechanical model of a joint», or «digital twin», as explained, is meant a multidimensional object (particularly two-dimensional or three-dimensional, and preferably three-dimensional) that is joined, that is to say movable in the same way as the modeled joint (and comprising all or part of the joint's degrees of freedom). The model optionally comprises deformable parts (corresponding in particular to fat or ligaments, which can be represented by volumetric meshes (for example finite element discretizations), and non-deformable parts (corresponding in particular to bones), which can be represented by 3D surface meshes.

The model is further advantageously defined by:
Input variables, particularly those known as «boundary conditions» that allow a posture to be defined (for example the extension/flexion and internal/external rotation angles for the knee; the activation level of a muscle, etc.). Mathematically, the biomechanical modeling space is defined as the space parameterized by these input variables, and the number of input variables defines the dimension of said space (for example in a 1-dimensional model, the flexion angle is the input variable, and in a 2-dimensional model, the flexion angle and the rotation angle are the input variables);
Configuration parameters:
   Constitutive laws: mechanical laws that govern the mechanical response of tissues to external stresses. Several types of laws can be considered depending on their relevance (targeted accuracy, realism, etc.);
   Biomechanical parameters: elasticity, Poisson's, friction, damping coefficients, etc. The values of these parameters can be deterministic (constant) or stochastic (variables, defined by a probability distribution);
Output data, in particular a deformed configuration of the model («the deformed shape»), constituting a simulation of said joint for said input variables, from which one can obtain the positions of characteristic anatomical points of said joint (also called anatomical points of interest, or more simply «landmarks») that can be associated with both non-deformable and deformable parts (points, curves, etc.), and the values of biomechanical metrics related to said landmark positions.

The characteristic anatomical points can, for example, be, in the case of the knee:
«Bone» anatomical points such as
   H (hip): center of the hip (center of the femoral head);
   A (ankle): ankle center (midpoint of the two malleoli);
   K (knee): knee center, also called femur center (central point of the femoral arch);
   T (tibia): tibia center
Anatomical points «bone tissue/soft tissue interface» such as
   insertions of the (internal and external) collateral ligaments;
   femoral and tibial insertions of the anterior and posterior cruciate ligaments.

The present invention will not be limited to any characteristic anatomical point, any metric, or generally any type of biomechanical model of a joint. Furthermore, different models may be used for different joints.

This method is implemented by a server 1 having data processing means 11 (typically a processor), and generally data storage means 12 (a memory, for example, a hard drive) and an interface 13 (for example, a screen, a keyboard, an input port, etc.).

Preferably, a medical imaging system 10 is also used for acquiring medical images of said joint.

This system 10 may be directly or indirectly (for example, via a network 20 such as the Internet) connected to said server 1 so that the latter is capable of receiving said medical images. The interface 13 of the server 1 may also serve as an interface for the system 10 (to control it and obtain the acquired medical images).

Said medical images are typically 3D volumetric images, possibly reconstructed from 2D sections, i.e., tomograms or «CT scans» (the system 10 is typically an X-ray scanner-CT (computed tomography)). Note that the system is not limited to a particular technology, and the system 10 could be an MRI, an ultrasound scanner, a PET scanner, etc.

### Method

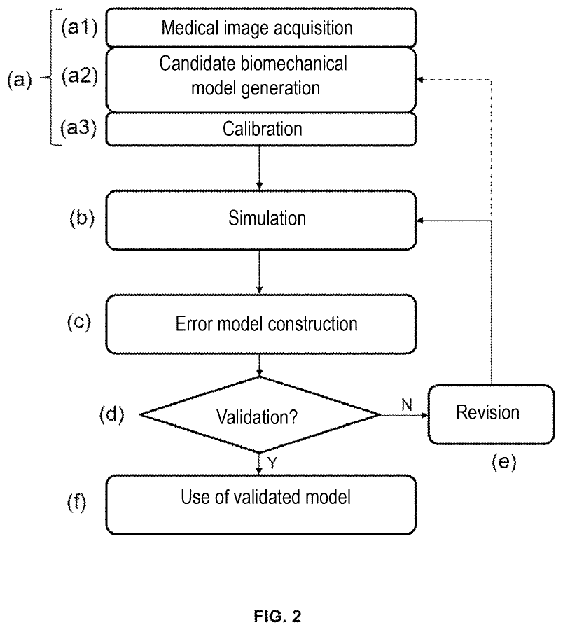

With reference to FIG. 2, the present method is implemented by the data processing means 11 of the server 1, and begins with a step (a) of obtaining a candidate biomechanical model of said joint and at least one first set (denoted E1) of medical images of said joint, called validation images. These images will be used to validate or reject the candidate biomechanical model. O

As will be seen, we further advantageously obtain a second set (denoted E2) of medical images of said joint, called initialization images, and, if necessary, a third set (denoted E3) of medical images of said joint, called calibration images. These images are used to generate said candidate biomechanical model, but alternatively, the method can be used to validate a model provided as is, and then only the first set is required. In any case, all the images are naturally of the same joint from the same patient.

Each image in each of these sets represents said joint in a posture, as explained above. To rephrase, a medical image is a snapshot of the joint in which it presents a particular posture among the set of possible postures, and thus each medical image is «associated» with the corresponding posture. Preferably, all medical images are associated with different postures, even if there may be some duplicates, and mathematically:
Either we have a discrete set of N_{P} postures for which a medical image has been acquired (denoted P_{acq}, with P_{acq} ⊂Pₐₗₗ, the latter theoretical set encompassing all postures that can be adopted by the joint of interest), and we can denote by Iᵢ the medical image associated with the i-th posture, with 1≤i≤Nₚ.
Or the posture is defined by one or more parameters with variable values (a flexion angle, for example), and we can directly denote by I(x1 . . . x_{d}) the medical image associated with the posture in which the parameters have the values x₁ . . . x_{d}. Pₐₗₗ is then the set of possible values of the vector [x₁ . . . x_{d}].

In all cases, a first set of postures (denoted P1, with P1⊂P_{acq}) can be defined as the set of postures associated with the medical images of the first set of medical images (validation images), and, where applicable, a second/third set of postures (denoted P2/P3, with P2/P3⊂P_{acq}) as the set of postures associated with the medical images of the second/third set of medical images (initialization/calibration images). Conversely, each image of the first set of medical images E1 represents said joint in a posture of the first set of postures P1, and, where applicable, each image of the second/third set of medical images E2/E3 represents said joint in a posture of the second/third set of postures P2/P3.

Preferably:
the first set of postures and the second set of postures are distinct (without intersection), i.e. P1∩P2=∅, which means that there is no validation image representing the joint in the same posture as an initialization image;
the first set of postures and the third set of postures are distinct (without intersection), i.e. P1∩P3=∅, which means that there is no validation image representing the joint in the same posture as a calibration image;
the second set of postures and the third set of postures are different (not identical) but not necessarily distinct (i.e., there may be a common posture, and therefore the same medical image, in the second and third sets of medical images), and we can even have P2 included in P3, as long as ∀P∈P3 such that P∉P2.

Indeed, it is understood that, for the validation to be as reliable as possible, images on the basis of which the candidate model was generated should not be reused as validation images.

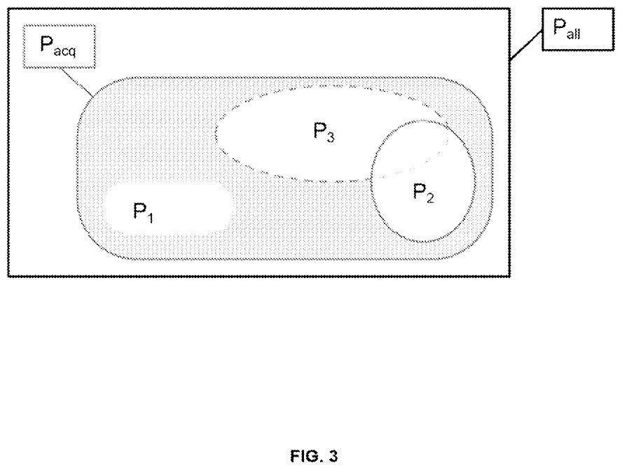

This is illustrated in FIG. 3: we see that the sets P1, P2, and P3 are all included in the set P_{acq} of postures for which an image has been acquired, itself included in the set Pₐₗₗ of possible postures, with the validation set P1 distinct from the initialization and calibration sets P2 and P3. The sets P1, P2, and P3 may moreover be as diverse as possible in said set P_{acq}, and particularly the first set P1. It is indeed desirable that the validation images in particular constitute a representative sample of the modeling space.

Preferably, the step (a) comprises sub-steps (a1) of obtaining (preferably by acquisition, by the medical imaging device 10) said first, second, and/or third set(s) of medical images of said joint; (a2) generating said candidate biomechanical model from said second set of medical images; and advantageously (a3) calibrating the candidate biomechanical model based on said third set of medical images.

Typically, the step (a1) consists of:
acquiring a large number of medical images (the set of acquired images being denoted E_{acq}, and recalling that P_{acq} is the set of postures associated with the medical images in this set E_{acq}) corresponding to various postures, in particular by asking the patient to move their joint, if possible over the full amplitude (for example full knee flexion/torsion),
then selecting, from this set E_{acq}, E1, E2, and/or E3, for example, by simply partitioning E_{acq}, or by first selecting, from the set P_{acq} of postures, the sets P1, P2, and/or P3, by diversifying as much as possible, then constructing E1, E2, and/or E3, respectively, as the sets of medical images associated with the postures in sets P1, P2, and/or P3.

The steps (a2) and, if applicable, (a3) aim to obtain the candidate biomechanical model. The step (a2) may be sufficient, but the step (a3) allows for the improvement of the generated model before use.

Said calibration can be seen as «fine-tuning» of a «provisional» model, in particular to refine certain model parameters, so as to significantly increase the chances of its validation.

Any technique known to those skilled in the art may be used for the steps (a2) and, if applicable, (a3); see, for example, the documents Towards Automatic Generation of Patient-Specific Knee Models by Elaheh Elyasi, Marek Bucki, Boubaker Asaadi, Daniel Elizondo, and Antoine Perrier, in Proceedings of the 20th Annual Meeting of the International Society for Computer-Assisted Orthopaedic Surgery, vol. 5, pp. 1-2, 66-68, 2022, or A fast and robust patient-specific Finite Element mesh registration technique: application to 60 clinical cases, Marek Bucki, Claudio Lobos, Yohan Payan, in Medical Image Analysis, vol. 3, pp. 303-17, 2010.

We will now be able to verify that the configurations produced at the output of the candidate model coincide with at least one validation image (and preferably a large number of images in the first set, or even all of them).

To do this, in a main step (b), the data processing means 11 implement at least one simulation of the candidate biomechanical model in which said joint presents the same posture as in said medical image of said first set of validation images E1. We will distinguish two implementation modes: a non-deterministic mode in which multiple simulations are performed, and a deterministic mode in which a single simulation is sufficient, which will be detailed later.

Generally speaking, simulation means putting the model into a state that mimics the validation image. More precisely, this simulation reproduces the posture in which the joint is represented in the validation image by applying appropriate input variable values, that is to say by defining boundary conditions for the model. Indeed, each medical image is a snapshot of the joint in a particular posture, whereas the model presents input variables and, in theory, can reproduce any posture from the set of possible postures Pₐₗₗ (and therefore the P_{acq} set it contains).

Then, in a following step (c), for at least one biomechanical metric of said joint (preferably for each) and for at least one validation image for which at least one simulation has been implemented (preferably for each validation image for which one or more simulations have been implemented), an error model of the candidate biomechanical model is constructed (for this joint of this patient) based on the values of the biomechanical metric(s) in said validation image and in the simulation(s) implemented for this validation image. It is recalled that these biomechanical metrics are typically linked to the positions of characteristic anatomical points (called «landmarks»), in particular, either directly to the positions of anatomical points in a given frame of reference, or to quantities calculated from these positions of characteristic anatomical points, in particular distances between two anatomical points or angles defined by three anatomical points, but other quantities such as volumes can be used.

For example:
in the case of bony anatomical points, the HKA (Hip-Knee-Ankle) angle in the frontal (radiological) plane, or in the sagittal plane, or even in 3D space can be taken as a metric;
in the case of anatomical points at the bone/self-tissue interface, the metric can be taken as the elongation distance (between the insertion points) of the collateral ligaments or cruciate ligaments, depending on the flexion angle.
By choosing the appropriate anatomical points (points at the ends of the bones), the metric can be taken as the length/width of the bones, the diameter of the femoral head, etc.

In all cases, typically:
if the biomechanical metrics are the positions of the anatomical points, their values are directly simulated;
if the biomechanical metrics are the distances/angles between the positions of the anatomical points or others, their values are calculated from the simulated values of the positions of the anatomical points.

The principle of validation consists of calculating predictions using the candidate model and using the images as ground truth to assess the accuracy of these predictions. We will see later, in each of the deterministic and non-deterministic cases, how this model can be constructed.

Finally, in a step (d), the candidate biomechanical model is validated or not based on the error model. We repeat that this is a validation for the joint of a patient, i.e., we validate that the biomechanical model specifically reproduces this joint of a patient with sufficient accuracy: the aim is not to globally validate a biomechanical model, but to ensure that it is correctly adapted to the joint of a patient in particular.

To do this, we advantageously predefine maximum uncertainty thresholds for each considered metric, and we check whether the uncertainty estimated by the error model is less than the maximum tolerable uncertainty. If the uncertainties are acceptable (i.e., sufficiently low) for all biomechanical metrics, then the validation is successful, and we then speak of a validated biomechanical model. Preferably, we calculate, for each posture (i.e., each validation image) and for each considered metric, a confidence interval on the value of said metric, and the candidate biomechanical model is rejected (not validated) if the amplitude of at least one given number (in particular one, but potentially more if a certain tolerance is accepted) of the calculated confidence intervals exceeds the said uncertainty threshold for the considered metric. Thus, a model can be validated for all or only a subset of the biomechanical metrics.

More precisely, for each biomechanical metric m considered for the step (d), by confidence interval (IC) of level p (in [0, 1]), we mean the narrowest interval such that the probability that the value of the predicted metric falls within this interval IC is p. The value of p is selected based on the criticality of the metric in therapeutic decision-making; for example, for critical metrics, p can be set to 0.95.

The «random» nature of a metric's value stems from two factors: 1) a possibly non-deterministic biomechanical model whose outputs, in particular, are not fixed values but are rather defined as probability distributions; and 2) a non-deterministic error model that affects each simulation result.

For each simulation, the probability density of the value of a biomechanical metric M as a random variable is estimated statistically, yielding a mean value and a variance. By assuming that M follows a normal distribution, its mean and variance are used to determine the minimum and maximum bounds of the confidence interval, given an expected confidence level p. The width of the IC is the difference between the upper and lower bounds of the interval, width=(max−min). If the width is large, then the uncertainty in M is high; if the IC is narrow, then the uncertainty is small. The smaller the uncertainty, the greater the confidence in the model's prediction. The candidate biomechanical models leading to excessive uncertainty (excessive IC width) should be rejected.

If the model is validated, it can simply be rendered on the interface 13, or used in a medical application, as discussed later.

Otherwise, the method may comprise an optional step (e) of revising the candidate biomechanical model if it is not validated, then repeating steps (b) to (d) based on the revised biomechanical model as a new candidate biomechanical model, and so on iteratively until a validated model is obtained or the modeling procedure is abandoned.

The step (e) may require reinitializing the biomechanical model, using a new initialization image and/or modifying parameters of the initialization procedure (such as for example the fineness of the finite element discretization of the deformable parts). In this case, this step (e) returns the protocol to step (a2) (see dotted arrow in FIG. 2). Alternatively, step (e) can be performed without modifying the model as initialized in step (a). In this case, step (e) consists of refining one or more model parameters (e.g., the elastic modulus of a ligament fiber) so that the model's behavior is more faithful to reality, and to do this, it is necessary to return to level (b) of the protocol (see the solid arrow in FIG. 2).

Note that step (e) can be systematically implemented if the candidate model is not validated, or only if the error remains limited. For example, maximum revision thresholds (larger than the maximum uncertainty thresholds) are predefined for each considered metric, and a check is made to see whether the error model is compatible with them (for example if at least the given number of calculated confidence intervals exceeds the metric's uncertainty threshold, but no calculated confidence interval exceeds the metric's revision threshold).

To summarize, in a preferred mode:
if the error (width of the confidence intervals) is less than the uncertainty thresholds→the model is validated.
if the error (width of the confidence intervals) is between the error thresholds and the revision thresholds→the model is revised.
if the error (width of the confidence intervals) is beyond the revision thresholds» the model is too erroneous to be revised, and the method is repeated, in particular by acquiring new sets of medical images, and a completely new candidate model is generated.

This method ensures that a validated model respects reality within a predefined accuracy corridor and can therefore be used, for example, to plan an operation or design a prosthesis.

The method may thus finally comprise a step (f) of using the validated biomechanical model in a medical application, in particular for a simulation such as implemented in step (b), this time in which said joint exhibits a target posture, for example, to estimate the values of said biomechanical metrics in this posture. Indeed, the validation ensures that the model can be used across the entire range of postures, including those outside the first, second, and third sets.

Note that step (f) may use said error model constructed in the step (c) to determine the maximum uncertainty in these estimated values of the biomechanical metrics (i.e., the accuracy corridor mentioned just before). The idea is to enable comprehensive risk management (taking into account, in particular, a «worst-case scenario»), which is essential in critical use of the biomechanical model, for example, before a surgical operation.

Note that in practice, at the end of the step (c), we obtain individualized error models, that is to say models associated with isolated validation images (and therefore postures in the modeling space). In order to extend error modeling to the entire biomechanical modeling space (the space defined by the input variables of the biomechanical model), it is possible to assume that the error model parameters vary continuously in this space and interpolate (N-linear interpolation according to the dimension of the error model, b-splines, or other).

Note that in the case of the presence of the step (f), the present method can be considered as a method for simulating a target posture of the joint (and generally for exploiting the biomechanical model in a medical application) rather than simply as a method for modeling the joint.

### Non-Deterministic Mode

In this non-deterministic mode, typically corresponding to a «fuzzy» biomechanical mode, the model outputs, including the metrics, are probabilistic, and therefore statistical processing must be implemented.

In step (b), for each validation image, a predetermined number N_{S} of simulations is advantageously implemented and a Monte Carlo type approach is preferably followed. The output is a collection of simulated values of said biomechanical metrics for the considered posture (that of the validation image).

In the case of a discrete collection of postures (but those skilled in the art will be able to transpose to the continuous case, see above), this collection is denoted {L(i,m,s)}, with i the posture index, with 0<i≤N_{P} (i.e. the validation image is Iᵢ∈E1), m the biomechanical metric index with 0<m≤N_{M} and s the simulation index with 0<s≤Ns.

In the validation image Iᵢ, the correct value of the biomechanical metric is known, and denoted R(i,m).

We can construct an error vector E (for example, 3-dimensional in the case where the metric is a position) with E(i,m,s)=L(i,m,s)−R(i,m), of which we can take a norm (to make it a scalar) with a norm function such as the 2-norm(but also the 1-norm or the infinite norm), i.e., norm(E(i,m,s)}=∥L{i,m,s)−R(i,m)∥.

The error model can be constructed in step (c) as a statistical model of E (vector quantity) or norm(E) (scalar quantity) based on all the samples corresponding to the various metrics and images.

In both cases, we can assume that this quantity follows a normal distribution (in view of the central limit theorem) such that:
for norm(E) (a scalar quantity), the mean and variance can be estimated conventionally,
for E (a vector quantity), the density function can be estimated, for example, by implementing a PCA (Principal Component Analysis).

We will now consider two examples of biomechanical metrics:
the distance between two anatomical points;
the angle formed by three anatomical points.

### Example of the Distance Between Two Anatomical Points

As explained above, we determine the two collections L1={L(i,1,s)} and L2={L(i,2,s)} corresponding respectively to the positions of each of the two anatomical points in the N_{S} simulations, and E1, E2 and/or norm(E1), norm(E2).

For each posture, we refer to statistical charts to calculate a predefined number of errors N_{E} for E1, E2, and/or norm(E1), norm(E2), following the distribution. Each error is then added to each simulated value to obtain a set of N_{S}*N_{E} realizations for the positions of each of the two anatomical points.

Since we want a distance here, we calculate all the distances corresponding to all possible pairs of realizations obtained for the positions of each of the two anatomical points, thus obtaining (N_{S}*N_{E})² distance values.

A new statistical analysis can be performed on all the distance values obtained (since in a perfect model we would always obtain the same value), in particular by estimating the mean and variance.

We can then calculate a confidence interval.

If for at least the given number of postures (in particular only one), the width of the confidence interval exceeds the maximum uncertainty threshold, the model is rejected/revised (see above).

### Example of the Angle Defined by Three Anatomical Points

As explained above, we determine the three collections: L1={L(i,1,s)}, L2={L(i,2,s)}, and L3={L(i,3,s)} corresponding respectively to the positions of each of the two anatomical points in the N_{S} simulations, and E1, E2, E3 and/or norm(E1), norm(E2), norm(E3).

For each posture, we then calculate a predefined number N_{E} of errors for E1, E2, E3 and/or norm(E1), norm(E2), norm(E3) following the distribution, and each error is added to each simulated value so as to obtain a collection of N_{S}*N_{E} realizations for the positions of each of the three anatomical points.

Since we want an angle here, we calculate all the angles corresponding to all possible triplets of the realizations obtained for the positions of each of the three anatomical points, thus obtaining (N_{S}*N_{E})³ angle values.

A new statistical analysis can be implemented on all the angle values obtained (since in a perfect model we would always obtain the same value), in particular by estimating the mean and variance.

A confidence interval can then be calculated.

If, for at least the given number of postures (in particular a single one), the width of the confidence interval exceeds the maximum uncertainty threshold, the model is rejected/revised (see above).

### Deterministic Mode

In this deterministic mode, typically corresponding to a biomechanical mode in which all parameters and laws have unique, well-defined values, the model outputs and therefore the corresponding metrics are unique.

In step (b), for each validation image, a single simulation is advantageously implemented. The output is a collection of simulated values of said biomechanical metrics for the considered posture (that of the validation image).

In the case of a discrete collection of postures (but those skilled in the art will know how to transpose this to the continuous case, see above), this collection is denoted {L(i,m)}, with i the index of the posture, with 0<i≤N_{P} (i.e., the validation image is Iᵢ∈E1), m the index of the biomechanical metric, with 0<m≤N_{M}.

In the validation image Iᵢ, the correct value of the biomechanical metric is known, and denoted R(i,m).

This time, we directly take an error norm (to make it a scalar) with a norm function such as the 2-norm (but also the 1-norm or the infinite norm), which we denote by maxE(i,m))=∥L(i,m)−R(i,m)∥.

Indeed, we have only one value per metric and per posture, so we cannot deduce a probability density. Thus, we consider the error observed at each posture to be the maximum error of the candidate model.

In other words, we assume a uniform distribution.

We will now again consider the two cases of biomechanical metrics:
the distance between two anatomical points
the angle formed by three anatomical points.

### Example of the Distance Between Two Anatomical Points

We only need to determine the two collections L1={L(i,1)}, L2={L(i,2)} corresponding respectively to the positions of each of the two anatomical points, which only comprise one value per validation image, and maxE(i,1), maxE(i,2).

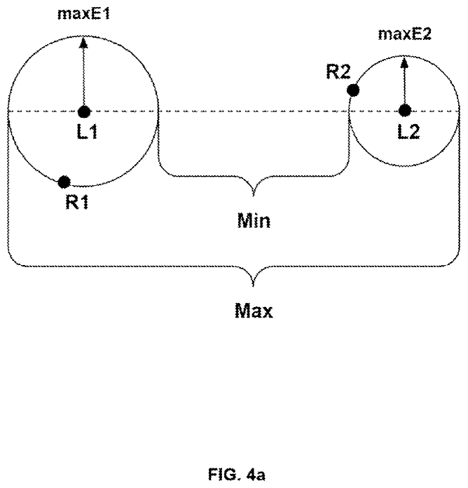

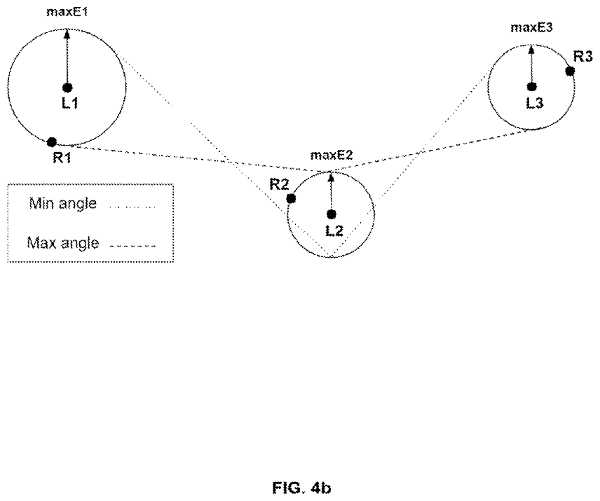

Referring to FIG. 4a, we can simply use geometric modeling to directly calculate the confidence interval, by representing said two anatomical points by two spheres with centers L(i,1) and L(i,2), and respective radii maxE(i,1) and maxE(i,2).

The bounds of the confidence interval then correspond to the minimum and maximum distances between two points on the two spheres:

$$max = d ( L ( i , 1 ) ; L ( i , 2 ) ) + max E ( i , 1 ) + max E ( i , 2 ) , and$$

$$min = d ( L ( i , 1 ) ; L ( i , 2 ) ) - max E ( i , 1 ) - max E ( i , 2 )$$

If, for at least the given number of postures (in particular, only one), the width of the confidence interval exceeds the maximum uncertainty threshold, the model is rejected/revised (see above).

### Example of the Angle Defined by Three Anatomical Points

We only need to determine the three collections L1={L(i,1)}, L2={L(i,2)}, L3={L(i,3)} corresponding respectively to the positions of each of the three anatomical points, which only comprise one value per validation image, and maxE(i,1), maxE(i,2), maxE(i,3).

Referring to FIG. 4b, we can again use geometric modeling to directly calculate the confidence interval, by representing the three anatomical points by three spheres with centers L(i,1), L(i,2), and L(i,3), and with respective radii maxE(i,1), maxE(i,2), and maxE(i,3). In the shown example, the vertex of the angle is arbitrarily the second anatomical point.

The bounds of the confidence interval then correspond to the minimum and maximum angles defined by three points on the three spheres. A numerical optimization is this time necessary to find these minimum and maximum angles.

If, for at least the given number of postures (in particular a single one), the width of the confidence interval exceeds the maximum uncertainty threshold, the model is rejected/revised (see above).

### Server

According to a second aspect, the invention concerns the server 1 for implementing the method according to the first aspect.

Thus, this server 1 comprises, as explained, at least data processing means 11 and a memory 12. The server may further comprise an interface 13 such as a screen. This is typically a server for modeling a joint of a patient.

The data processing means 11 are configured to implement steps consisting of:
Obtaining a candidate biomechanical model of said joint and a first set of medical images of said joint, representing said joint in a posture from a first set of postures, called validation images;
For at least one medical image of said first set of medical images of said joint, implementing at least one simulation of the candidate biomechanical model in which said joint exhibits the same posture as in said medical image of said first set of images;
Constructing an error model of the candidate biomechanical model by comparing, for at least one biomechanical metric of said joint and for at least one medical image of said first set of medical images of said joint for which at least one simulation has been implemented, the values of said biomechanical metric in said medical image and in the simulation(s) implemented for said medical image;
Validating or not the candidate biomechanical model based on the error model.

According to a third aspect, the invention proposes a system comprising said server 1, as well as a connected medical imaging device 10 (via the network 20).

The data processing means 11 are then further configured to:
obtain from the medical imaging device 10 said first set of medical images of said joint, as well as a second set of medical images of said joint, representing said joint in a posture from a second set of postures (and potentially a third set of medical images of said joint, representing said joint in a posture from a third set of postures);
generate said candidate biomechanical model from the second set of medical images; and preferably
calibrate said candidate biomechanical model based on said third set of medical images.

The data processing means 11 may also be configured to:
revise the candidate biomechanical model if it is not validated; or
use the validated biomechanical model by implementing at least one simulation of this model in which said joint exhibits a target posture.

### Computer Program Product

According to a fourth and fifth aspect, the invention concerns a computer program product comprising code instructions for executing (on the data processing means 11 of the server 1) a method according to the first aspect of modeling a joint of a patient, as well as storage means readable by computer equipment (for example, the data storage means 12 of the server 1) on which this computer program product is located.

1. A method for modeling a joint of a patient, the method being characterized in that it comprises the implementation, by data processing means of a server, the steps of:
(a) obtaining a candidate biomechanical model of the joint and a first set of medical images of the joint, representing the joint in a posture from a first set of postures, called validation images;
(b) for at least one medical image of the first set of medical images of the joint, implementing at least one simulation of the candidate biomechanical model in which the joint exhibits the same posture as in the medical image of the first set of images;
(c) constructing an error model of the candidate biomechanical model by comparing, for at least one biomechanical metric of the joint and for at least one medical image of the first set of medical images of the joint for which at least one simulation has been implemented, the values of the biomechanical metric in the medical image and in the simulation(s) implemented for the medical image;
(d) validating or not the candidate biomechanical model based on the error model.

2. The method according to claim 1, wherein step (a) comprises sub-steps (a1) of obtaining the first set of medical images of the joint, as well as a second set of medical images of the joint, representing the joint in a posture of a second set of postures; and (a2) generating the candidate biomechanical model from the second set of medical images.

3. The method according to claim 2, wherein the first set of postures and the second set of postures are distinct.

4. The method according to claim 2, wherein step (a1) also comprises obtaining a third set of medical images of the joint, representing the joint in a posture of a third set of postures; step (a) comprising a sub-step (a3) of calibrating the candidate biomechanical model based on the third set of medical images.

5. The method according to claim 3, wherein step (a1) also comprises obtaining a third set of medical images of the joint, representing the joint in a posture of a third set of postures; step (a) comprising a sub-step (a3) of calibrating the candidate biomechanical model based on the third set of medical images, and wherein the first set of postures and the third set of postures are distinct; and the second set of postures and the third set of postures are different but not necessarily distinct.

6. The method according to claim 2, wherein step (a1) comprises the acquisition of the second set of medical images, and where appropriate a third set of medical images, by a medical imaging device.

7. The method according to claim 1, wherein step (d) comprises the calculation, for the biomechanical metric(s) and the medical image(s) of the first set, of a confidence interval on the value of the biomechanical metric from the error model, and the candidate biomechanical model is not validated if a width of at least a given number of the calculated confidence intervals exceeds an uncertainty threshold of the metric.

8. The method according to claim 7, wherein the values of the biomechanical metric in the medical image and in the simulation(s) implemented for the medical image are calculated in step (c) based on the positions of characteristic anatomical points of the joint in the medical image and in the simulation(s) implemented for the medical image.

9. The method according to claim 8, wherein there is a plurality of characteristic anatomical points of the joint, and the biomechanical metric is a distance between two of the characteristic anatomical points of the joint or an angle defined by three of the characteristic anatomical points of the joint.

10. The method according to claim 1, comprising a step (e) of revising the candidate biomechanical model if it is not validated, then repeating at least steps (b) to (d) based on the revised biomechanical model as a new candidate biomechanical model.

11. The method according to claim 1, comprising a step (f) of using the validated biomechanical model, comprising implementing at least one simulation of the validated biomechanical model in which the joint exhibits a target posture.

12. The method according to claim 11, wherein step (f) comprises using the error model to assess a level of uncertainty on the result of the simulation of the validated biomechanical model.

13. The method according to claim 1, wherein either the candidate biomechanical model is non-deterministic, and in step (b) for each medical image of the first set a plurality of simulations is implemented; or the candidate biomechanical model is deterministic, and in step (b) for each medical image of the first set a single simulation is implemented.

14. A server (1) for modeling a joint of a patient, wherein it comprises data processing means (11) configured to:
obtain a candidate biomechanical model of the joint and a first set of medical images of the joint, representing the joint in a posture of a first set of postures, called validation images;
for at least one medical image of the first set of medical images of the joint, implementing at least one simulation of the candidate biomechanical model in which the joint exhibits the same posture as in the medical image of the first set of images;
constructing an error model of the candidate biomechanical model by comparing, for at least one biomechanical metric of the joint and for at least one medical image of the first set of medical images of the joint for which at least one simulation has been implemented, the values of the biomechanical metric in the medical image and in the simulation(s) implemented for the medical image;
validating or rejecting the candidate biomechanical model based on the error model.

15. A system comprising a server according to claim 14 and a medical imaging device, the data processing means being further configured to:
obtain from the medical imaging device the first set of medical images of the joint, as well as a second set of medical images of the joint, representing the joint in a posture of a second set of postures;
generate the candidate biomechanical model from the second set of medical images.

16. A computer program product comprising code instructions for executing a method according to claim 1 for modeling a joint of a patient, when the program is executed on a computer.

17. A storage medium readable by computer equipment on which is recorded a computer program product comprising code instructions for executing a method according to claim 1 for modeling a joint of a patient.
